(19) United States
(12) Patent Application Publication
(10) Pub. No.: US 2003/0229048 A1
(43) Pub. Date: Dec. 11, 2003

(54) Methods and compositions for inhibition of irritation by disaccharide and metal ions

(76) Inventors: Dange Veerapaneni, San Diego, CA (US); Kazutaka Inoue, Kanagawa (JP); Takaaki Yoshinaga, Saga (JP); Munehiko Hirano, Saga (JP); Shuji Sato, Kanagawa (JP)

Correspondence Address: BROMBERG & SUNSTEIN LLP, 125 SUMMER STREET, BOSTON, MA, 02110-1618, US

(21) Appl. No.: 10/426,941

(22) Filed: Apr. 30, 2003

Related U.S. Application Data
(60) Provisional application No. 60/376,573, filed on Apr. 30, 2002.

(51) Int. Cl. A61K 31/7012; A61K 31/557

(52) U.S. Cl. 514/53; 424/401; 514/161; 514/573; 514/570

(57) ABSTRACT

Compositions for reducing an adverse skin reaction caused by a skin-irritating agent or skin sensitizing agent by treatment with a disaccharide and a metal ion, as well as methods for reducing an adverse skin reaction caused by a skin-irritating or skin-sensitizing agent, and methods for treating an inflammatory response to at least one inflammatory agent, by application of a disaccharide and/or metal ion composition.

### CROSS REFERENCE TO RELATED APPLICATIONS

This application claims priority from U.S. provisional application No. 60/376,573, filed Apr. 30, 2002, the contents of which are hereby incorporated herein by reference.

### TECHNICAL FIELD AND BACKGROUND ART

The present invention relates to compositions and methods for preventing or treating an adverse response to an inflammatory agent, or a skin sensitizing or skin-irritating agent using a formulation including a disaccharide and/or a metal ion.

### SUMMARY OF THE INVENTION

In a general embodiment of the present invention, there is provided a composition comprising an adverse skin reactive agent or skin sensitizing agent and an adverse skin reaction preventing, reducing or controlling agent comprising an effective amount of a disaccharide and a metal ion. In accordance with an embodiment of the present invention, the skin-sensitizing agent may be a therapeutic agent, a metal, a fragrance, a cosmetic, a textile, pollen, a pesticide, a plastic, and combinations thereof.

Another embodiment of the present invention the skin sensitizing agent of the composition maybe a therapeutic agent including an antibiotic, an antiviral, an analgesic and analgesic combination, an anorexic, an anti-arthritic, an anti-asthmatic, an anti-coagulant, an anti-convulsant, an antidepressant, an anti-diabetic, an anti-diarrheal, an antihistamine, an anti-inflammatory agent, an anti-migraine agent, an anti-motion sickness preparation, an anti-nauseant, an anti-neoplastic, an anti-parkinsonism drug, an anti-pruritic, an antipsychotic, an anti-pyretic, an anti-spasmodic, an anti-cholinergic, a sympathomimetic, a xanthine derivative, a cardiovascular agent, an anti-arrhythmic, an anti-hypertensive, a vasodilator, a central nervous acting agent, a cough and cold preparation, a decongestant, a diagnostic, a hormone, a hypnotic, a muscle relaxant, a parasympatholytic, a parasympathomimetic, a psychostimulant, a sedative, a weight control and appetite suppressive drug, an a tranquilizer.

In yet another embodiment of the present invention, the skin sensitizing agent of the claimed composition may be an anti-inflammatory agent including methyl salicylate, acetylsalicylic acid, sodium salicylate, choline salicylate, choline magnesium salicylate, diflunisal, salflex, salicylamide, salsalate, disalcid, trolamine salicylate, trisilate, ketoprofen, prostaglandin, flurbiprofen, diclofenac, indomethacin, piroxicam, and ibuprofen.

Another embodiment of the present invention provides a composition comprising an adverse skin reactive agent or skin sensitizing agent and an adverse skin reaction preventing, reducing or controlling agent comprising an effective amount of a disaccharide and a metal ion, wherein at least one monosaccharide in the disaccharide is selected from a hexose, a pentose, a tetrose and a triose. More particularly, the at least one monosaccharide in the disaccharide is selected from glyceraldehyde, dihydroxyacetone, erythrose, threose, erythrulose, ribose, arabinose, xylose, lyxose, ribulose, xylulose, allose, altrose, glucose, mannose, gulose, idose, galactose, talose, psicose, fructose, sorbose, tagatose, deoxyribose, quinovose, rhamnose, and fucose, and more particularly, the disaccharide in the composition is a trehalose.

Yet another embodiment provides a composition comprising an adverse skin reactive agent or skin sensitizing agent and an adverse skin reaction preventing, reducing or controlling agent comprising an effective amount of a disaccharide and a metal ion, wherein the metal ion is a divalent metal ion, for example, zinc, magnesium, manganese, copper, iron, aluminum, calcium, cobalt, silver, and cadmium. More particularly, the metal ion is in the form of a metal oxide, for example zinc oxide, magnesium oxide, manganese oxide and the like.

Another embodiment of the present invention provides a method for treating an adverse skin reaction of the skin in a subject to the presence of at least one of a skin-sensitizing or a skin-irritating agent comprising the steps: providing an adverse skin reaction preventing, reducing or controlling agent comprising a disaccharide and a metal ion in a formulation; and topically administering an effective amount of the formulation to the subject so as to prevent, reduce or control the adverse skin reaction.

An another embodiment, the skin-irritating agent is a compound including water, cleansers, alkalis, acids, oils, organic solvents, oxidizing agents, and combinations thereof.

Alternatively, the skin-sensitizing agent is selected from the group consisting of therapeutic agents, metals, fragrances, cosmetics, textiles, pollen, pesticides, plastics, and combinations thereof, and the adverse skin reaction preventing, reducing or controlling agent is administered transdermally; or, the skin-sensitizing agent is a therapeutic agent and the method further comprising administering the adverse skin reaction preventing, reducing or controlling agent and the therapeutic agent from a transdermal patch.

Yet another embodiment in accordance with the present invention provides a method of treating an inflammatory response in a subject to the presence of at least one inflammatory agent comprising the steps: providing an inflammatory preventing, reducing or controlling agent comprising a disaccharide and a metal ion in a formulation; and administering an effective dose of the formulation to the subject, so as to prevent, reduce or control the inflammatory response.

Still another embodiment provides a method of preventing an inflammatory response in a subject according to the third general embodiment, further comprising administering the effective dose via topical administration including transdermal patch or tape formulation, subcutaneous administration, parentereal administration, intravenous administration, instramuscular administration, and oral administration.

Another embodiment provides a method of preventing an inflammatory response according to the third general embodiment, wherein the inflammatory response is evidenced by a stimulation of cytokines, growth factors, and chemokines, for example, by a stimulation of interleukin-1α and tumor necrosis factor-α.

Yet another embodiment provides a method of treating an inflammatory response comprising the steps: providing an inflammatory preventing, reducing or controlling agent comprising a disaccharide and a metal ion in a formulation; and topically administering an effective amount of the formulation to the subject so as to prevent, reduce or control the adverse skin reaction.

In particular there is presented a method for treating an inflammatory response wherein the inflammatory response is a response to the presence of an agent selected from the group consisting of methyl salicylate, acetylsalicylic acid, sodium salicylate, choline salicylate, choline magnesium salicylate, diflunisal, salflex, salicylamide, salsalate, disalcid, trolamine salicylate, and trisilate.

Still another embodiment provides a method of treating an adverse reaction of the skin in a subject to the presence of at least one of a skin-sensitizing or a skin-irritating agent comprising the steps: providing an adverse skin reaction preventing, reducing or controlling agent comprising a disaccharide in a formulation; and administering an effective amount of the disaccharide formulation to the adverse reactive site of the subject in a suitable carrier so as to prevent, reduce or control the adverse skin reaction. More particularly, the method may further comprise administering the effective dose of the disaccharide formulation via topical administration including transdermal patch or tape formulation, subcutaneous administration, parentereal administration, intravenous administration, instramuscular administration, and oral administration.

Another embodiment provides a method of treating an adverse reaction of the skin in a subject to the presence of at least one of a skin-sensitizing or a skin-irritating agent comprising the steps: providing an adverse skin reaction preventing, reducing or controlling agent comprising a metal ion in a formulation; and administering an effective amount of the metal ion formulation to the adverse reactive site of the subject in a suitable carrier so as to prevent, reduce or control the adverse skin reaction. More particularly, the method may further comprise administering the effective dose of the metal ion formulation via topical administration including transdermal patch or tape formulation, subcutaneous administration, parentereal administration, intravenous administration, instramuscular administration, and oral administration.

Yet another embodiment provides a method of treating an inflammatory condition in a subject to the presence of at least one inflammatory agent comprising the steps: providing an inflammatory preventing, reducing or controlling agent comprising a disaccharide in a formulation; and administering an effective amount of the disaccharide formulation to the subject in a suitable carrier so as to prevent, reduce or control the inflammation. More particularly, the method may further comprise administering the effective dose of the disaccharide formulation via topical administration including transdermal patch or tape formulation, subcutaneous administration, parentereal administration, intravenous administration, instramuscular administration, and oral administration.

Still another embodiment includes a composition effective in treating an adverse skin reaction comprising an adverse skin-reaction preventing, reducing or controlling agent comprising a therapeutically effective amount of a disaccharide and a metal ion.

Alternatively, there is provided a composition effective in treating an inflammatory response comprising an inflammatory preventing, reducing or controlling agent comprising a therapeutically effective amount of a disaccharide and a metal ion.

### BRIEF DESCRIPTION OF THE DRAWINGS

The foregoing features of the invention will be more readily understood by reference to the following detailed description, taken with reference to the accompanying drawings, in which:

FIG. 1 shows the structures of Trehalose A and Trehalose B.

FIG. 2 is a graph showing the effects of Trehalose A and B on IL-1α.

FIG. 3 is a graph showing the effects of Trehalose C on IL-1α.

FIG. 4 is a graph showing the effects of Zinc Oxide on IL-1α.

FIG. 5 is a graph showing the concentration effects of Zinc Oxide on IL-1α.

FIG. 6 is a graph showing the effects of Trehalose B with Zinc Oxide on IL- 1α.

FIG. 7 is a graph showing the effects of Zinc Oxide on Keratinocyte cell viability.

### DETAILED DESCRIPTION OF SPECIFIC EMBODIMENTS

Definitions. As used in this description and the accompanying claims, the following terms shall have the meanings indicated, unless the context otherwise requires:

“Inflammatory preventing agent” as used herein, means, any agent, molecule, compound, formulation, etc. capable of preventing an inflammatory response to an inflammatory agent, as indicated by repeated post-exposure measurement of non-elevated levels of cytokines, growth factors, and chemokines, particularly interleukin-1α and tumor necrosis factor-α chemokines, observed after administration of an inflammatory preventing agent with an inflammatory agent, as compared to the levels of cytokines, growth factors, and chemokines measured after administration of no inflammatory agent or measured after administration of the inflammatory agent alone.

“Inflammatory reducing agent” as used herein, means, any agent, molecule, compound, formulation, etc. capable of reducing an inflammatory response to an inflammatory agent, as indicated by post-exposure measurement of reduced levels of cytokines, growth factors, and chemokines, particularly interleukin-1α and tumor necrosis factor-α chemokines, observed after administration of an inflammatory reducing agent with an inflammatory agent, as compared to the levels of cytokines, growth factors, and chemokines measured after administration of the inflammatory agent alone.

“Inflammatory controlling agent” as used herein, means, any agent, molecule, compound, formulation, etc. capable of controlling an inflammatory response to an inflammatory agent, as indicated by repeated post-exposure measurement of unchanged levels of cytokines, growth factors, and chemokines over time, particularly interleukin-1α and tumor necrosis factor-α chemokines, observed after administration of an inflammatory controlling agent with an inflammatory agent, as compared to the levels of cytokines, growth factors, and chemokines measured over time after administration of the inflammatory agent alone.

“Adverse skin reaction preventing agent as used herein, means, any agent, molecule, compound, formulation, etc. capable of preventing an adverse skin reaction to a skin-sensitizing agent or skin-irritating agent, as indicated by objective and subjective evidence of non-itchy, non-irritated, non-painful, non-swollen, non-red, non-blotchy, non-inflamed, or otherwise normal looking and feeling skin observed and/or reported after administration of an adverse skin reaction preventing agent with a skin-sensitizing or skin-irritating agent, as compared to the objective and subjective evidence regarding skin condition observed/reported after administration of no skin-sensitizing or skin-irritating agent or measured after administration of the skin-sensitizing or skin-irritating agent alone.

“Adverse skin reaction reducing agent” as used herein, means, any agent, molecule, compound, formulation, etc. capable of reducing an adverse skin reaction to a skin-sensitizing or skin-irritating agent, as indicated by objective and subjective evidence of non-itchy, non-irritated, non-painful, non-swollen, non-red, non-blotchy, non-inflamed, or otherwise normal looking and feeling skin observed and/or reported after administration of an adverse skin reaction preventing agent with a skin-sensitizing or skin-irritating agent, as compared to the objective and subjective evidence regarding skin condition observed/reported after administration of the skin-sensitizing or skin-irritating agent alone.

“Adverse skin reaction controlling agent” as used herein, means, any agent, molecule, compound, formulation, etc. capable of controlling an adverse skin reaction to a skin-sensitizing or skin-irritating agent, as indicated by sustained objective and subjective evidence of non-itchy, non-irritated, non-painful, non-swollen, non-red, non-blotchy, non-inflamed, or otherwise normal looking and feeling skin observed and/or reported after administration of an adverse skin reaction controlling agent with a skin-sensitizing or skin-irritating agent, as compared to the sustained objective and subjective evidence regarding skin condition observed/reported after administration of the skin-sensitizing or skin-irritating agent alone.

Epidermal Keratinocytes play an active role in the irritation of primary contactirritancy and contact-hypersensitivity reaction in the skin through the synthesis and production of proinflammatory mediatory like cytokines, growth factors and chemokines, including IL-1α, and TNF-α. Methyl Salicylate (MS) is an active ingredient in oil of wintergreen, which is used, in various over-the-counter topical preparations indicated to relieve musculo-skeletal pains and aches. The potential systemic toxicity and the risk of skin irritation of the topical preparations is well documented in the literature and has been attributed to diverse actions of the compounds like MS on human cells.

Previously, we have shown that MS stimulates release of the primary cytokines, especially IL-1α, in epidermal keratinocytes and anti-irritants like ethacrynic acid (ETA) inhibited the IL-1α release by MS. These results suggested that cytokine release by epidermal keratinocytes could be used as a marker for predicting the skin irritation potential of topically applied MS. The current study examined the effectiveness of commercially available trehalose, a disaccharide, to block the MS stimulated IL-1α release. We used three commercially available trehalose preparations: namely, trehalose A, B, and C. The three trehalose preparations used in this study differed in that one contained trace amounts of metal ions (trehalose A) and the other two were highly purified forms (trehalose B and trehalose C) which do not contain any metal ions. Results showed that only trehalose A was capable of inhibiting MS induced IL-1α release compared to trehalose B and C, which had no effect. Chemical analysis of these trehalose preparations confirmed that the only difference between the three preparations was the presence of metal ions in the trehalose A preparation. The analysis also indicated that zinc was the most abundant metal ion found in the trehalose A preparation. Hence, the ability of zinc oxide to inhibit MS induced IL-1α was tested. Results showed that ZnO was able to block MS induced IL-1α production as effectively as trehalose A, thus revealing a novel anti-irritant effect of these compounds in epidermal keratinocytes. These findings suggest that trehalose A or zinc oxide could be used in transdermal drug delivery systems to prevent skin irritation.

Methods:

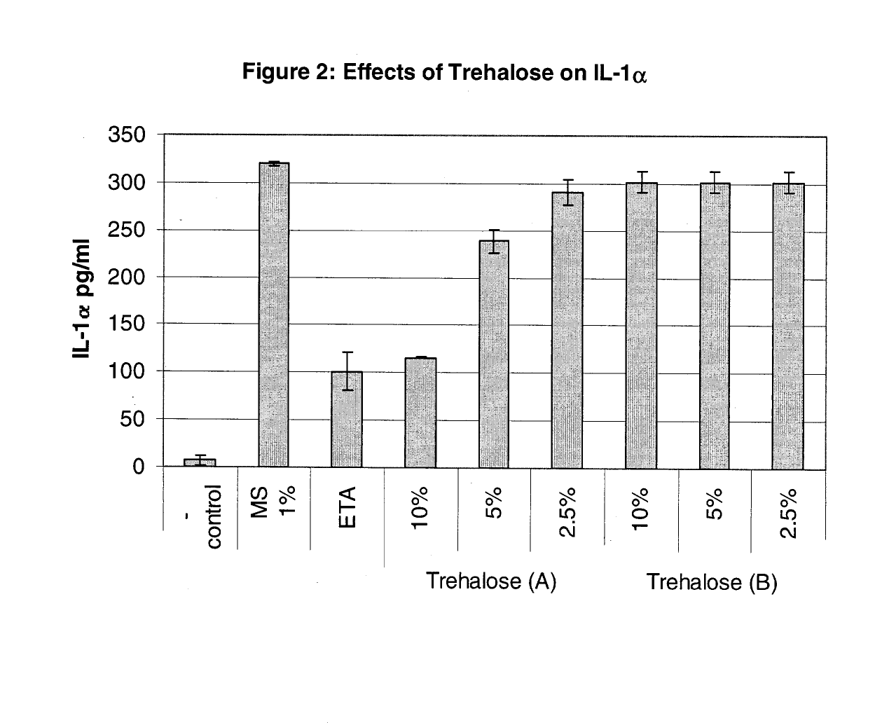

Reagents methyl salicylate, ethacrynic acid, trehalose A, B, and C were obtained from Sigma-Aldrich (St. Louis, Mo.). Trehaloses were dissolved in keratinocyte media. Methyl salicylate was diluted in keratinocyte media. Ethacrynic acid was dissolved in DMSO. Keratinocyte cells were grown to 100% confluency as per the instructions given by ATCC. Then the cells were trypsinized to dislodge cells and spun at 3000 rpm for 3 min. The supernatant was aspirated and the cells were re-suspended in fresh media at a density of 1.0×10⁶ cells per mL. The cell suspension was the pipetted into a 96-well plate (200 ul of per well) and cells were incubated overnight at 37° C. The next day morning media was aspirated, and 100 uL of fresh medium containing methyl salicylate (2%) was added to all the wells except for the (−) control wells. No MS was included in the (−) control wells. Then 100 uL of medium containing the various test reagents were added to the wells so that the final concentrations were as indicated in FIGS. 2-7. The 96-well plate was then incubated. After incubation for four hours at 36° C., the supernatant from the wells was transferred to centrifuge tubes and centrifuged to remove the cell debris. Then the presence of IL-1α was determined using ELISA kit (R&D systems, Inc.) as per the manufacturer's specifications. The cells remaining in the wells were treated with pre-warmed media containing MTT solution and incubated for four hours. Then the formazan product (metabolic product of MTT) in the wells was solubilized and the absorbance was measured using a plate reader.

Results:

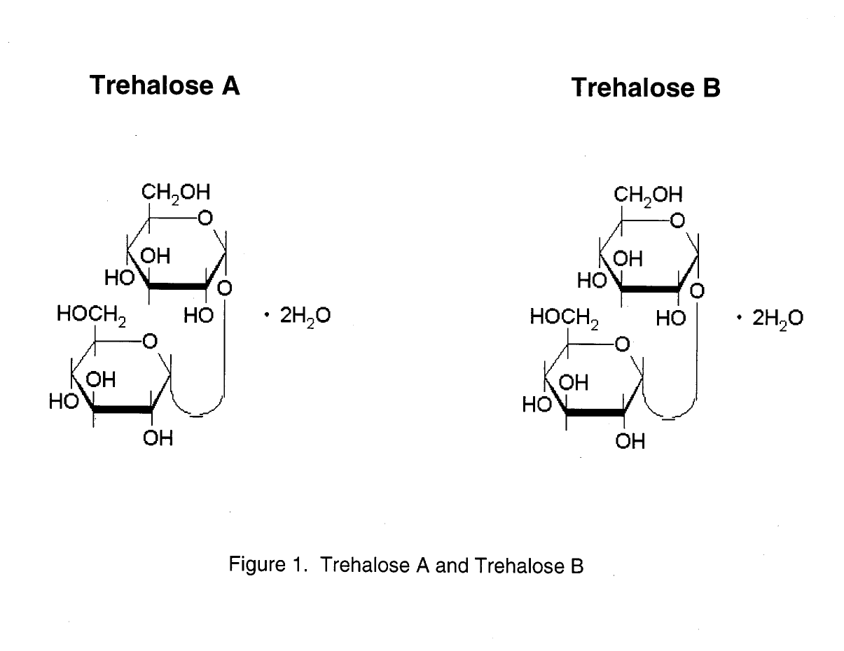

The three different varieties of trehalose preparations used in this study (trehalose A, B, and C) had the same chemical formula and structure as well as the same optical isomerism (see FIG. 1). The only difference between the preparations is the manner in which they were purified. The purification process removed metal ions from trehalose B and trehalose C preparations, whereas the trehalose A preparation showed trace amounts of zinc ions.

Human keratinocytes were stimulated with MS in the absence or the presence of trehalose compounds, to find an optimal concentration of trehalose at which IL- 1α production is reduced by half compared to the positive control.

FIG. 2 shows the effects of higher concentrations of Trehalose A and B on IL-1alpha concentrations. Ethacrynic Acid (ETA) at 0.16%, and 10% Trehalose A exhibit over 50% reduction in IL-1α levels compared to MS 1% (positive control). 5% Trehalose A has much lower levels of reduction while 2.5% Trehalose A has no effect. At 10% Trehalose A the reduction in IL-1α is almost as great as the reduction of 0.16% Ethacrynic acid. Trehalose B at any of these concentrations has no effect. Higher concentrations than 10% of Trehalose A, B or C were not chosen because of the difficulty involved in dissolving the samples. At low concentrations of 0.1-2.5%, neither trehalose A nor B showed any effect.

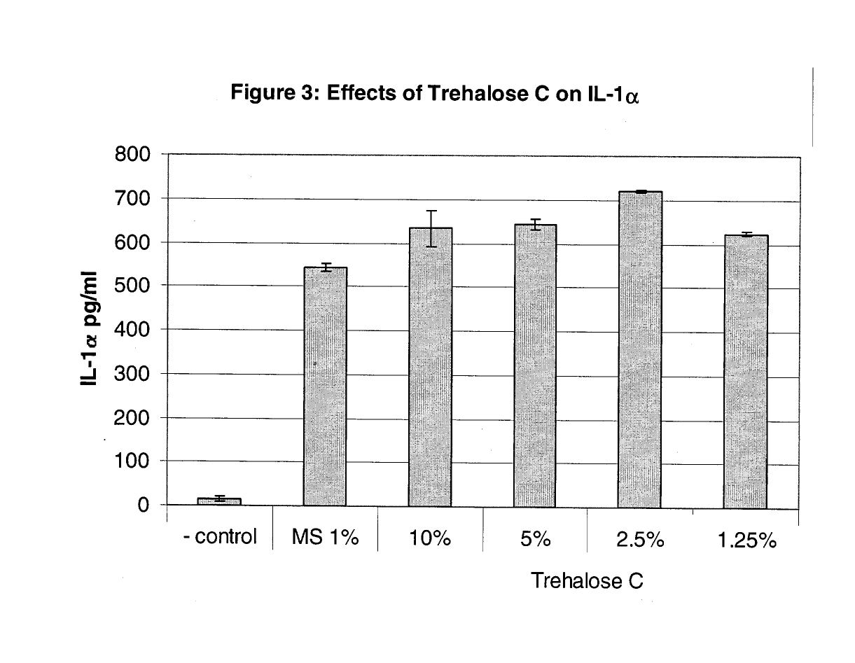

FIG. 3 shows the effects of Trehalose C on IL-1α levels in Keratinocyte cells stimulated by 1% Methyl Salicylate. In contrast, trehalose B and C had no effect at any concentrations that were tested (see FIGS. 2 and 3). Since the inhibition of IL-1α occurred only with trehalose A and the difference between the trehaloses tested was only the presence of zinc in trehalose A, experiments were performed to determine whether the inhibitory effect of trehalose A was caused by zinc or the trehalose A compound alone.

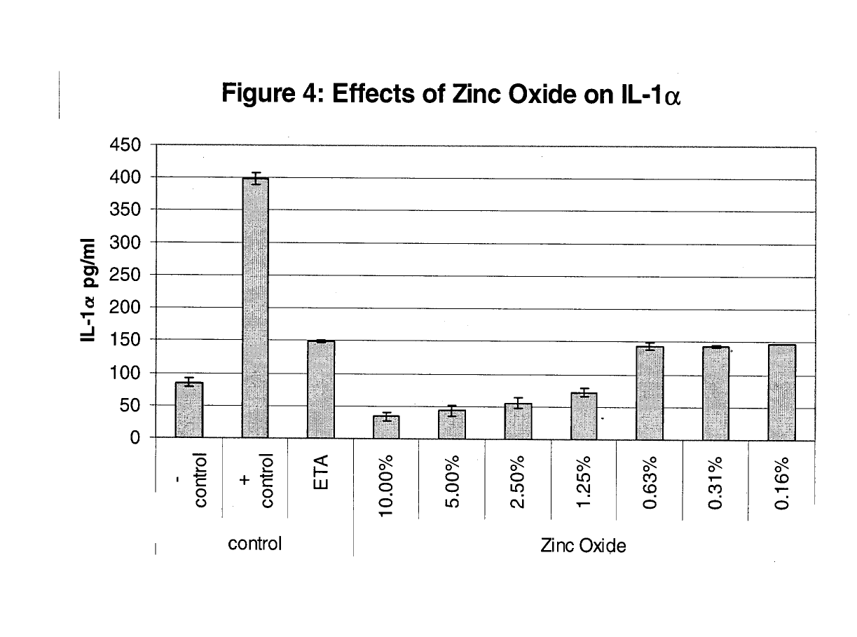

Experiments were performed using zinc oxide alone, or in combination with trehalose A, B, or C. FIG. 4 shows the effects of high concentrations of Sample A (Zinc Oxide alone) on IL-1α alpha levels in Keratinocyte cells stimulated with 1% Methyl Salicylate. The − control, + control and Ethacrynic acid (0.16%) wells are as previously shown in FIGS. 1-3. When we incubated the cells with high concentrations of zinc oxide, there was a dramatic reduction of MS stimulated IL-1α levels, but the cells were not active. Even at high to medium concentrations the IL-1α levels were significantly reduced indicating possible antiirritancy potential of Zinc Oxide. Sample A shows a large decrease in IL-1α levels at the concentration levels 10%-1.25%. At concentrations of 0.63% and below Sample A shows an inhibition of IL-1α similar to the inhibition shown by ETA (Ethacrynic Acid 0.16%). The main difference between Trehalose A and B is the presence of the metal ion zinc, which has been removed from Trehalose B preparations.

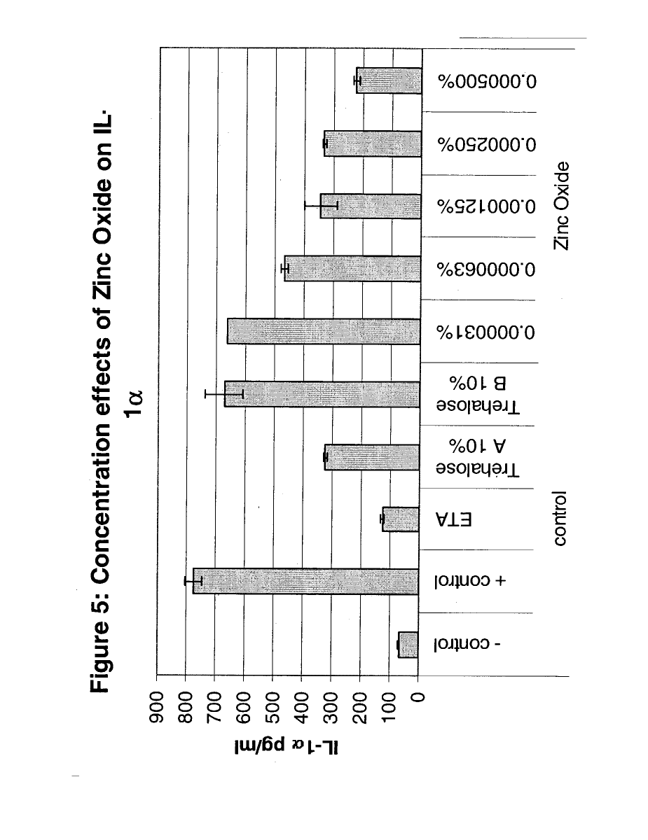

Further studies showed that even at lower concentrations zinc oxide showed a dose dependent decrease in methyl salicylate induced IL-1α levels in keratinocyte cells. FIG. 5 shows the effects of ETA (Ethacrynic acid), Trehalose A and B, and Sample A (Zinc Oxide) on IL-1α levels in Keratinocyte cells stimulated by 1% Methyl Salicylate. The − control, + control, and ETA (Ethacrynic acid 0.16%) wells show IL-1α concentrations as previously shown. Trehalose A 10% shows greater than 50% inhibition of IL-1α alpha concentration. Trehalose B has no effect on IL-1α levels in Keratinocyte cells stimulated with 1% Methyl Salicylate. Sample A (Zinc Oxide) shows a significant dose dependent decrease in IL-1α concentrations. At very low levels of Zinc Oxide concentration the reduction of IL-1α in Keratinocytes induced by Methyl Salicylate is shown in a dose dependent manner. At concentrations as low as 0.000125% Zinc Oxide clearly shows a greater than 50% reduction in IL-1α concentrations indicating possible anti-irritancy potential.

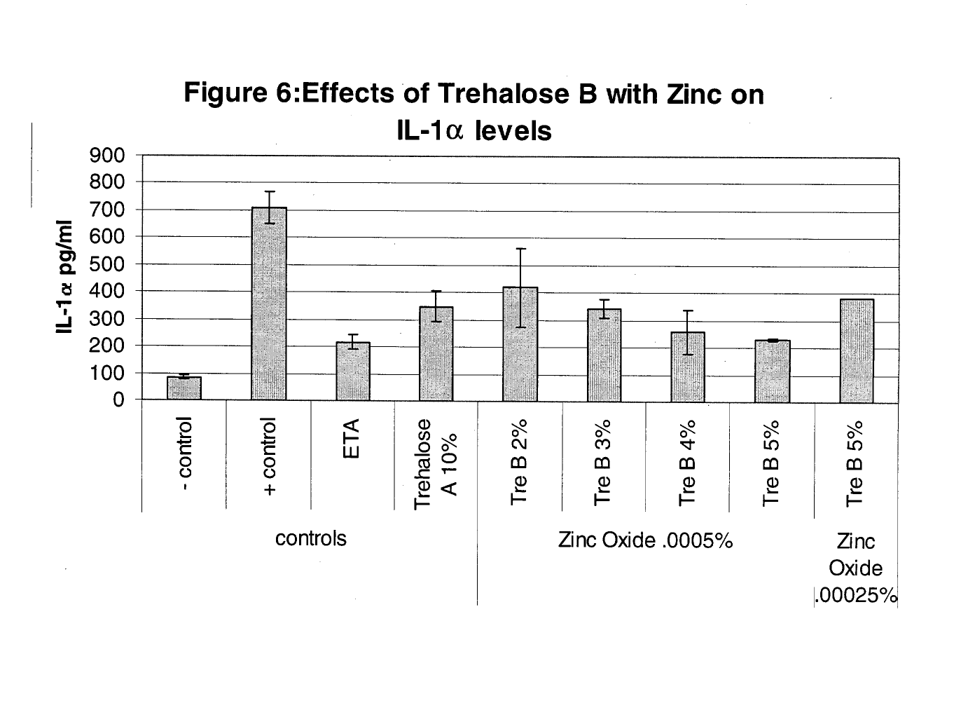

These results suggest that the inhibition was dependent on the presence of zinc in trehalose A. In order to confirm these results, we used a combination of zinc oxide and trehalose B (which is inactive). FIG. 6 shows the effects of Trehalose B combined with Zinc Oxide on IL-1α levels in Keratinocyte cells. The controls for this experiment show data that is consistent with previous experiments. Zinc Oxide in combination with Trehalose B shows a large dose dependent decrease in IL-1α concentrations induced by Methyl Salicylate. As Zinc Oxide levels are decreased there is an increase in IL-1alpha concentrations at the same Trehalose B concentration indicating. that Trehalose B along with Zinc lower IL-1α concentrations. Thus, interestingly, trehalose B in combination with zinc oxide was able to inhibit MS stimulated IL-1α levels.

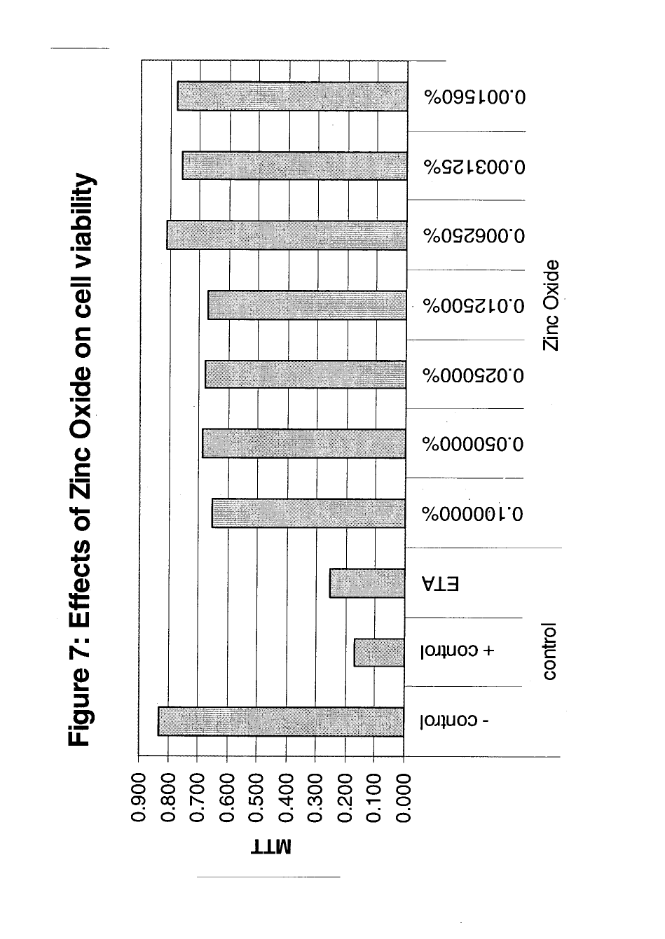

We also tested the cytotoxicity of zinc oxide on keratinocyte cells using the MTT assay as described in the Methods section. FIG. 7 shows the effects of Zinc Oxide on MTT levels in Keratinocyte cells. The − control, + control and ETA (Ethacrynic acid 0.16%) wells are as previously shown. Zinc Oxide shows no effect on MTT concentration levels in Keratinocyte cells, as indicated by healthy cells metabolizing MTT into the purple dye formazan salt. Thus, the concentrations at which ZnO inhibited the MS stimulated IL-1α release had no effect on cell viability.

What is claimed is:

1. A composition comprising:
an adverse skin-reactive agent, skin-irritating agent, or skin-sensitizing agent and an adverse skin-reaction preventing, reducing or controlling agent comprising a therapeutically effective amount of a disaccharide and a metal ion.

2. A composition according to claim 1, wherein the skin-sensitizing agent is selected from the group consisting of therapeutic agents, metals, fragrances, cosmetics, textiles, pollen, pesticides, plastics and combinations thereof.

3. A composition according to claim 2, wherein the therapeutic agent is an agent including an antibiotic, an antiviral, an analgesic and analgesic combination, an anorexic, an antiarthritic, an anti-asthmatic, an anticoagulant, an anticonvulsant, an antidepressant, an anti-diabetic, an antidiarrheal, an antihistamine, an anti-inflammatory agent, an antimigrane agent, an anti-motion sickness preparation, an antinauseant, an antineoplastic, an antiparkinsonism drug, an antipruritic, an antipsychotic, an antipyretic, an antispasmodic, an anticholinergic, a sympathomimetic, a xanthine derivative, a cardiovascular agent, an antiaarrythmic, an antihypertensive, a vasodilator, a central nervous acting agent, a cough and cold preparation, a decongestant, a diagnostic, a hormone, a hypnotic, a muscle relaxant, a parasympatholytic, a parasympathomimetic, a psychostimulant, a sedative, a weight control and appetite suppressive drug, an a tranquilizer.

4. A composition according to claim 3, wherein the anti-inflammatory agent is an agent including methyl salicylate, acetylsalicylic acid, sodium salicylate, choline salicylate, choline magnesium salicylate, diflunisal, salflex, salicylamide, salsalate, disalcid, trolamine salicylate, trisilate, ketoprofen, prostaglandin, flurbiprofen, diclofenac, indomethacin, piroxicam, and ibuprofen.

5. A composition according to claim 1, wherein at least one monosaccharide in the disaccharide is selected from a hexose, a pentose, a tetrose and a triose.

6. A composition according to claim 1, wherein at least one monosaccharide in the disaccharide is selected from erythrose, threose, erythrulose, ribose, arabinose, xylose, lyxose, ribulose, xylulose, allose, altrose, glucose, mannose, gulose, idose, galactose, talose, psicose, fructose, sorbose, tagatose, deoxyribose, quinovose, rhamnose, and fucose.

7. A composition according to claim 1, wherein the disaccharide is a trehalose.

8. A composition according to claim 1, wherein the metal ion is a divalent metal ion.

9. A composition according to claim 8, wherein the divalent metal ion is zinc.

10. A composition according to claim 1, wherein the metal ion is in the form of a metal oxide.

11. A composition according to claim 10, wherein the metal oxide is zinc oxide.

12. A method for treating an adverse skin reaction of the skin in a subject to the presence of at least one of a skin-sensitizing or a skin-irritating agent comprising the steps:
providing an adverse skin reaction preventing, reducing or controlling agent comprising a disaccharide and a metal ion in a formulation; and
topically administering a therapeutically effective amount of the formulation to the subject so as to prevent, reduce or control the adverse skin reaction.

13. A method according to claim 12, wherein the skin-irritating agent is a compound including water, cleansers, alkalis, acids, oils, organic solvents, oxidizing agents, and combinations thereof.

14. A method according to claim 12, wherein the skin-sensitizing agent is selected from the group consisting of therapeutic agents, metals, fragrances, cosmetics, textiles, pollen, pesticides, plastics, and combinations thereof.

15. A method according to claim 12, wherein the adverse skin reaction preventing, reducing or controlling agent is administered transdermally.

16. A method according to claim 15, wherein the skin-sensitizing agent is a therapeutic agent, the method further comprising administering the adverse skin reaction preventing, reducing or controlling agent and the therapeutic agent from a transdermal patch.

17. A method of treating an inflammatory response in a subject to the presence of at least one inflammatory agent comprising the steps:
providing an inflammatory preventing, reducing or controlling agent comprising a disaccharide and a metal ion in a formulation; and
administering an effective dose of the formulation to the subject, so as to prevent, reduce or control the inflammatory response.

18. A method of treating an inflammatory response in a subject according to claim 17, further comprising administering the effective dose via topical administration including transdermal patch or tape formulation, subcutaneous administration, parentereal administration, intravenous administration, instramuscular administration, and oral administration.

19. A method of treating an inflammatory response according to claim 17, wherein treatment of the inflammatory response is evidenced by a reduction in the stimulation of cytokines, growth factors, and chemokines relative to the stimulation of cytokines, growth factors and chemokines observed in the absence of administration of an inflammatory preventing, reducing or controlling agent.

20. A method of treating an inflammatory response according to claim 19, wherein treatment of the inflammatory response is evidenced by a reduction in the stimulation of interleukin-1α and tumor necrosis factor-α chemokines relative to the stimulation of cytokines, growth factors and chemokines observed in the absence of administration of an inflammatory preventing, reducing or controlling agent.

21. A method of treating an inflammatory response according to claim 17 wherein the inflammatory response is in response to the presence of an agent selected from the group consisting of methyl salicylate, acetylsalicylic acid, sodium salicylate, choline salicylate, choline magnesium salicylate, diflunisal, salflex, salicylamide, salsalate, disalcid, trolamine salicylate, and trisilate.

22. A method of treating an adverse reaction of the skin in a subject to the presence of at least one of a skin-sensitizing or a skin-irritating agent comprising the steps:
providing an adverse skin reaction preventing, reducing or controlling agent comprising a disaccharide in a formulation; and
administering an effective amount of the disaccharide formulation to the adverse reactive site of the subject in a suitable carrier so as to prevent, reduce or control the adverse skin reaction.

23. A method of treating an adverse skin reaction in a subject according to claim 22, further comprising administering the disaccharide formulation via topical administration including transdermal patch or tape formulation, subcutaneous administration, parentereal administration, intravenous administration, instramuscular administration, or oral administration.

24. A method of treating an adverse reaction of the skin in a subject to the presence of at least one of a skin-sensitizing or a skin-irritating agent comprising the steps:
providing an adverse skin reaction preventing, reducing or controlling agent comprising a metal ion in a formulation; and
administering an effective amount of the metal ion formulation to the adverse reactive site of the subject in a suitable carrier so as to prevent, reduce or control the adverse skin reaction.

25. A method of treating an adverse skin reaction in a subject according to claim 22, further comprising administering the metal ion formulation via topical administration including transdermal patch or tape formulation, transdermal administration, subcutaneous administration, parentereal administration, intravenous administration, instramuscular administration, or oral administration.

26. A method of treating an inflammatory response in a subject to the presence of at least one inflammatory agent comprising the steps:
providing an inflammatory preventing, reducing or controlling agent comprising a disaccharide in a formulation; and
administering an effective amount of the disaccharide formulation to the subject in a suitable carrier so as to prevent, reduce or control inflammation.

27. A method of treating an inflammatory response in a subject according to claim 26, further comprising administering the disaccharide formulation via topical administration including transdermal patch or tape formulation, subcutaneous administration, parentereal administration, intravenous administration, instramuscular administration, or oral administration.

28. A composition effective in treating an adverse skin reaction comprising:
an adverse skin-reaction preventing, reducing or controlling agent comprising a therapeutically effective amount of a disaccharide and a metal ion.

29. A composition effective in treating an inflammatory response comprising:
an inflammatory preventing, reducing or controlling agent comprising a therapeutically effective amount of a disaccharide and a metal ion.
